(19) United States
(12) Patent Application Publication
(10) Pub. No.: US 2015/0259370 A1
(43) Pub. Date: Sep. 17, 2015

(54) INTEGRATED PROCESS EXTRACTION OF PINEAPPLE BIOMASS INTO FIBERS AND NATURAL PRODUCTS

(71) Applicant: Ecotech Development LLC, North Palm Beach, FL (US)

(72) Inventors: Marco A. Baez-Vasquez, North Palm Beach, FL (US); Betty Putilin, North Palm Beach, FL (US)

(73) Assignee: Ecotech Development LLC, North Palm Beach, FL (US)

(21) Appl. No.: 14/205,213

(22) Filed: Mar. 11, 2014

(51) Int. Cl. C07H 1/08; D21H 11/00; C12N 9/48

(52) U.S. Cl. CPC C07H 1/08; C12N 9/48; D21H 11/00

(57) ABSTRACT

The invention relates to a method for disassembling and extracting process for the sustainable production of natural fibers and several natural bio-based products obtained from pineapple plant biomass. The embodiment of the present invention comprises a simultaneous mechanical hydrodynamic disruption and homogenization processing, a combination of extracting disruptive fractionating action that has the capacity to crack and breakdown thoroughly plant cells and plant tissues from different parts of the pineapple plant to unlock biofibers and multiple natural components at cytologic level and plant tissues.

### FIELD OF THE INVENTION

The present invention relates to methods and products obtained from plants; and more particularly to a method of extracting multiple components, including fibers, active bio-catalytic enzymes and several natural bio-organic compounds from pineapple plants.

### BACKGROUND OF THE INVENTION

Rising global populations have lead to an increasing demand for energy, food, shelter and natural, green and renewable raw materials. As petroleum and petroleum derivatives maybe reaching saturation and the ever-growing environmental pressure caused by the widespread consumption of petroleum based fuels, or petrochemicals, a shift into the development of biodegradable and environmentally acceptable materials from natural resources is increasing. With this comes an increasing demand to develop substitutes that replace petroleum based materials with renewable natural materials that reduce pollution and minimize environmental footprint.

One such focus is on the use of sustainable and renewable materials. Pineapple (Ananas comosus) belongs to the Bromeliaceae family. It is a very popular plant worldwide because of the palatable pleasant fruit that it produces. The first native plant varieties originated from South America and are now widely cultivated in the great majority of the tropical and subtropical countries. Given the widespread growth, pineapple plants have gained attention as a renewable source for the production of chemical, natural products, food additives and biofuels. A variety of natural products can be extracted from pineapple residual plants. For example, the pineapple plant is a very important source of natural virgin fibers that exhibit high specific strength, stiffness and hygroscopic properties. The superior mechanical properties of pineapple leaf fibers are associated with high cellulose content, up to 85% to 90%, and comparatively low microfibrillar angle. Due to the unique properties exhibited by pineapple leaf fiber, they can be used as excellent potential reinforcement in biocomposite matrices and functional biomaterials.

In general, the processing of pineapple begins with the harvesting of the pineapple fruit and/or collecting of the fruit for the subsequent packaging process. Once the fruit collection is complete, the remaining pineapple plant is typically left on the ground for subsequent disposal processes. In order to clear and prepare the ground for the next cultivation cycle, pineapple growers often use herbicides such as N,N′-dimethyl-4,4′-bipyridinium dichloride, to aid in the process. Such herbicides are applied onto the surface of the remaining pineapple plant. Microbial cell degradation digests the remaining plant tissue, followed by the pineapple plant field burning and final mechanical grinding up for complete elimination.

While an effective means for eliminating the waste, field burning enhances the release of multiple fine particles and/or air pollutants such as carbon compounds, nitrogen oxides (NOx), Sulfur Oxides or Dioxides (Sox), Carbon Monoxide (CO) and methane (CH₄), formate (HCOO), acetate ions (CH₃COO), oxalyate ions (C₂O₂-4), sulfur dioxide (SO₂), nitrates (NO₃) and so forth. In addition to the use of chemicals, the field burning process involves production of green house gases due to the herbicide treated plant burning process. In addition, slow organic decomposition of the plants on the ground causes the production of insects which affect dairy farms and livestock of adjacent farmland. The present invention addresses some of the issues involved in the processing of pineapples by providing for methods that exhibit a significant capacity to utilize the pineapple plant biomass waste left at the cultivation ground after the fruit has been harvested. The remaining plant biomass represents an agricultural residue and a renewable biomass feedstock generated immediately after the fruit has been collected.

The present invention helps to introduce sustainable practices for biomass waste management and pollution prevention by means of eliminating the massive use of herbicides. The present invention is adapted to process most of the pineapple plant biomass, thereby eliminating the requirement of landfill space, reducing the use of expensive and contaminant traditional disposal methods which use the combination of chemical, biological, combustion, and mechanical processes to decompose the pineapple plant biomass after fruit harvesting. In addition to waste management and pollution prevention, a system and method designed to create natural products from the pineapple plant can address other issues, such as the economic and/or environmental impacts relating to the Stable Fly (Stomoxys calcitrans).

Issues such as outbreaks and/or parasite infection tend to increase due to pineapple waste accumulation. For example, during 2009 and 2011 in Central American countries, a 68% increase by about in outbreaks and attacks by Stable Fly was observed, particularly near pineapple fields. Stable Fly exhibit strong reproduction cycles on plant pineapple waste and the problem becomes more intense during high humidity and heavy rain periods. As a result of the high frequency fly outbreaks, livestock such as cows exhibit a dramatic reduction in milk yields (up to 50%), growth and weigh gain. These effects on the animal can be translated into economic losses for livestock farmers. It has been estimated that an animal may be prevented fro gaining as much as 600 grams per day if continuously attacked by the Stable Fly, i.e. for 2 hours. Therefore, the elimination of this parasite benefits cattle growers by securing higher production yields for meat and dairy.

Accordingly, the present invention could provide multiple economical and sustainable environmental benefits by preventing the use of herbicide-chemical-burning-mechanical disposal process and providing pineapple growers a mechanism to save millions of dollars through fast and economical means to prepare the ground for the next pineapple cultivation cycle. In addition to the above, the present invention will bring economical and sustainable environmental benefits for the cattle and dairy producers by disrupting the Fly Stable cycle and eliminating attacks on dairy and livestock farms.

### SUMMARY OF THE INVENTION

The present invention describes an innovative process for the extraction of pineapple biomass components, including fibers, active biocatalytic enzymes and several natural bio-organic compounds. Such materials are extracted using a process which includes the disassembling extraction of Pineapple plant agricultural waste, defined as any portion of the plant but the fruit of the plant as well as any waste from processing the plant. The process further includes downstream steps that exhibit a direct interdependence, i.e. each subsequent step in the processing relies on previous steps to form a processing cascade, during the extraction of natural substances, biocatalysts and commercial biofiber products from Pineapple plant biomass.

In a preferred embodiment, innovative method includes provides a single and integrated extraction of pineapple plant biofibers, multiple natural products and functional derivatives from the tissues of the pineapple plant biomass components. The present invention, therefore, enables a fast, integrated and cost-effective extraction of for example, dietary fiber for human consumption, sulfydril endopeptidases Bromelain enzyme complex, and other hydrolases including carbohydrases, fiber for paper production, functional starchy flour used in food and industrial applications, xylose from the hydrolytic pretreatment of fiber, xylitol for low caloric natural sweeteners and lumen for organic fertilizer, compost or animal feed; in addition fibers can be further extracted to produce microfibers and nanofiber for multiple industrial applications.

Preferably, the process is adapted to provide a minimal steps resulting in disassembling of the pineapple plant biomass to extract various components or constituents using a combination of mechanical, thermo-mechanical and chemical procedures in order to obtain biofibers, natural products and derivatives of commercial relevance. An embodiment of the present invention comprises a simultaneous mechanical hydrodynamic disruption and homogenization processing, a combination of extracting disruptive fractionating action that has the capacity to crack and breakdown plant cells and plant tissues from many different parts of the pineapple plant, thereby unlocking biofibers and multiple natural components at cytologic level and plant tissues. In the disassembling process it is possible to fractionate to homogeneity and obtain consistent uniform fractions. In the embodiment of the present invention the disassembling extracting process comprises hydrodynamic abrasion processes. A series of extractive products are obtained from the disassembling extraction of the pineapple plant biomass. The pineapple biomass was subjected to an extraction process and the treated material is subjected processes to separate the water soluble fiber material, including water soluble sugars, proteins and complex carbohydrates.

The pineapple biomass disassembling extraction process in accordance with the present invention can be used to as a single process to isolate various components from the pineapple plant using two or more parts of the plant. Alternatively, the pineapple biomass disassembling extraction process can be adapted to provide processes for isolating constituents from only one part of the pineapple plant. Accordingly, in one embodiment of the invention, a method for isolating and extracting a plurality of constituents from a pineapple plant comprises: obtaining a pineapple plant; separating said pineapple plant into at least two portions, one first portion containing pineapple plant stems and one second portion containing pineapple plant leaves, wherein each of said portions are processed to produce constituents from said pineapple plant; isolating a plurality of constituents from each of said first portion contain pineapple plant stems and said second portion containing pineapple plant leafs. The initial steps provide for a first stem separation fraction and a first leaf separation fraction. The process further includes steps to further process the first separation fractions, thereby isolating additional components from the first stem or leaf.

In an alternative embodiment, a method for isolating and extracting a plurality of constituents from the stems of a pineapple plant comprises: obtaining stems from a pineapple plant; subjecting said stems from a pineapple plant to a first set of procedures to form at least two first leaf separation fractions; isolating a plurality of constituents from each said first leaf separation fractions. The process further includes steps to further process the first separation fractions, thereby isolating additional components from the first stem or fractions.

In an alternative embodiment, a method for isolating and extracting a plurality of constituents from the leaves of a pineapple plant comprises: obtaining leaves from a pineapple plant; subjecting said leaves from a pineapple plant to one or more processes to form a plurality of first leaf separation fractions; isolating a plurality of constituents from each said first leaf separation fractions. The process further includes steps to further process the first separation fractions, thereby isolating additional components from the first leaf fractions.

Accordingly, it is an objective of the present invention to teach an integrated process for extraction of pineapple biomass into fibers and natural products.

It is a further objective of the present invention to teach fibers and natural products obtained from pineapple.

It is yet another objective of the present invention to teach fibers and natural products obtained from a process of extraction from a pineapple.

It is a still further objective of the invention to teach the extraction of pineapple biomass fibers, active biocatalytic enzymes and natural bio-organic compounds.

It is a further objective of the present invention to teach the extraction of pineapple biomass fibers, active biocatalytic enzymes and natural bio-organic compounds using a one step process which includes the disassembling extraction of Pineapple plant agricultural residues.

It is yet another objective of the invention to teach the extraction of pineapple biomass fibers, active biocatalytic enzymes and natural bio-organic compounds using the pineapple stem.

It is yet another objective of the invention to teach the extraction of pineapple biomass fibers, active biocatalytic enzymes and natural bio-organic compounds using the pineapple fruit pulp and core.

It is yet another objective of the invention to teach the extraction of pineapple biomass fibers, active biocatalytic enzymes and natural bio-organic compounds using the pineapple crown leaves.

It is yet another objective of the invention to teach the extraction of pineapple biomass fibers, active biocatalytic enzymes and natural bio-organic compounds using the pineapple leaves.

It is yet another objective of the invention teach the extraction of pineapple biomass fibers, active biocatalytic enzymes and natural bio-organic compounds using the pineapple fruit peel.

It is a still further objective of the invention to teach the extraction of pineapple biomass fibers, active biocatalytic enzymes and natural bio-organic compounds using the pineapple stem, fruit pulp and core, fruit peel, crown leaves and leaves.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1A is a flow chart illustrating various end products or substituent, such as biofibers, natural products and derivatives, isolated and obtained from the extraction process in accordance with the present invention;

FIG. 1B is a flow chart illustrating the general steps to obtaining end products or plant constituents, such as biofibers, natural products and derivatives, isolated and obtained from the extraction process in accordance with the present invention;

FIG. 2 illustrates a pineapple plant, showing several of the major structural components of a typical plant;

FIG. 3 is a bar graph illustrating the bromelain activity in different pineapple varieties, including Cayenne Lisa, MD2, Manzana, and Perolera;

FIG. 4 illustrates the effects of pH variation on residual bromelain activity;

FIG. 5 is a bar graph representing the total monosaccharide production during in vitro digestibility test of lumen from pineapple natural fibers.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

The present invention is directed towards a process for extracting various products from a pineapple plant, and is refereed to generally as a pineapple biomass disassembling extraction process 10. The pineapple biomass disassembling extraction process 10 involves disassembling and extracting processes that utilizes the agricultural waste or residues of the pineapple plant biomass and fruit as a raw material to extract various components. The pineapple biomass disassembling extraction process 10 may include using stems only, leaves only, or combinations of stems and leaves as a starting point. Accordingly, the processing cascade may include stems for isolating components from the stems, leaves, or combinations of stems and leaves. The extraction process is completed using, in combination one or more, hydro-mechanical, thermo-mechanical and extractive chemical procedures to obtain long, short, micro, nano-biofibers and natural products of commercial relevance. The extractants mainly contain cellulose, hemicellulose, xylose, pectin, functional starches, chlorophyll, complex carbohydrates, natural fibers, natural enzymes, dietary fiber and other bioorganic derivatives and nutraceutical compounds. As will be described in greater detail, the pineapple plant may be separated into stems and leaves. The leaves may undergo a delamination process in which the stems are processed so that portions of the plants may be stripped away to form individual components, forming soluble components, semi-solid components, and solid components. As part of the same process or cascade, or as a separate individual cascade, the stems may undergo a maceration process to form a solid component and a liquid component. Each of these initial round processing components can further be processed to form additional components.

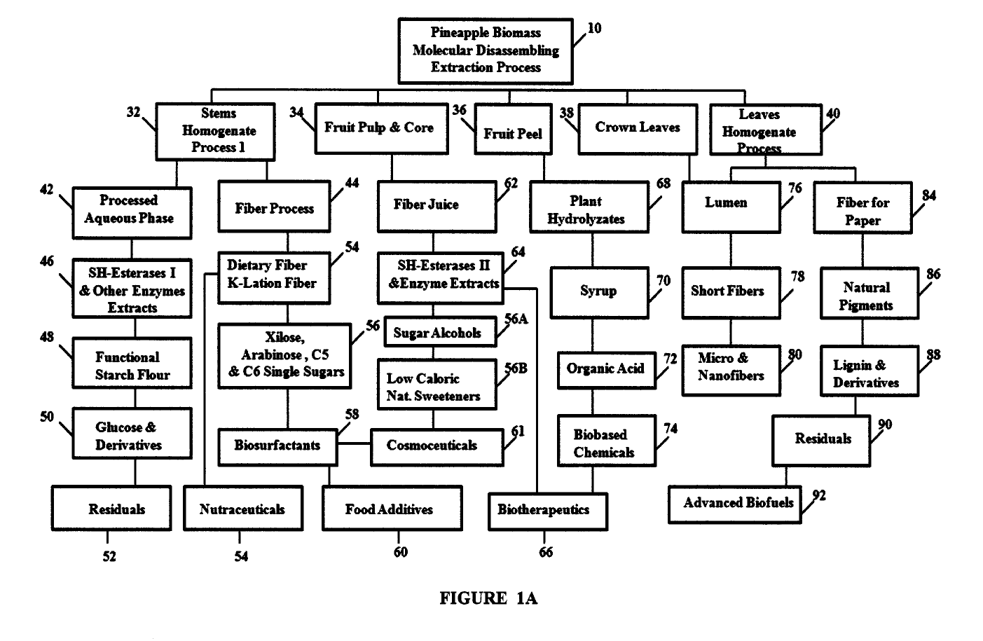

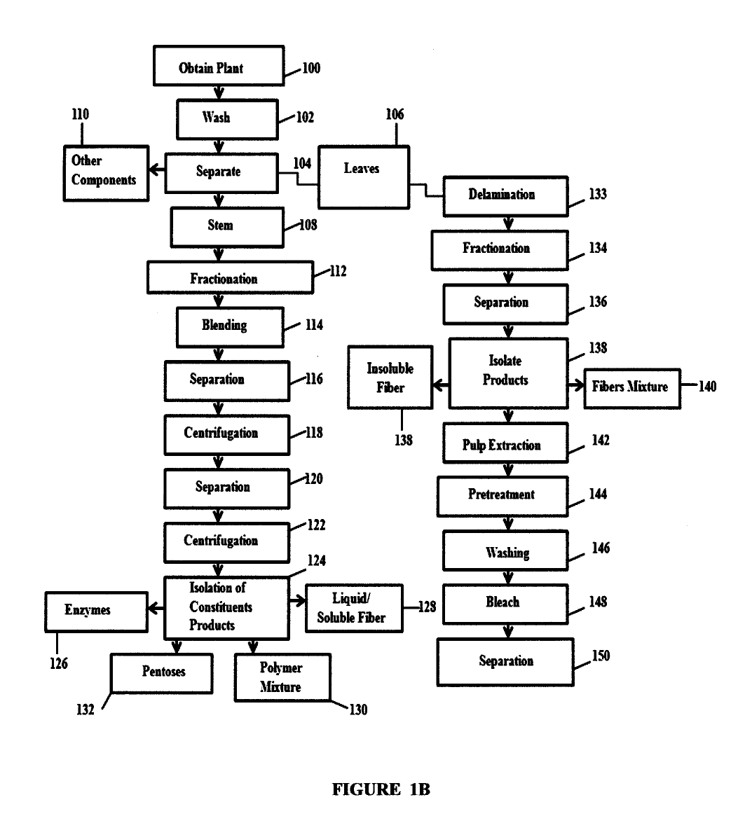

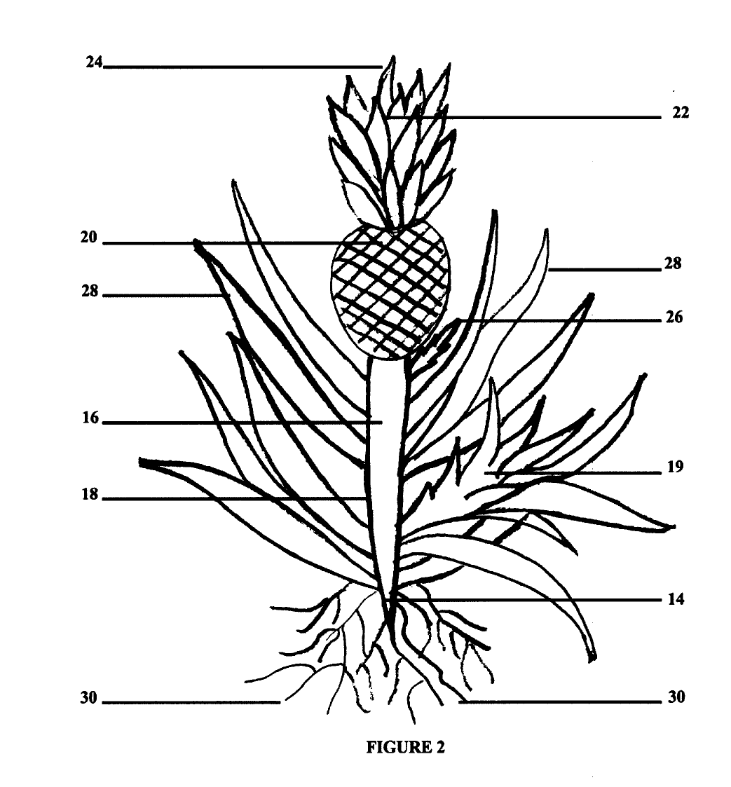

Referring to FIG. 1A, a flow chart illustrating various end products, such as biofibers, natural products and derivatives, isolated and obtained from the extraction process in accordance with the present invention is shown. The pineapple biomass disassembling extraction process 10 utilizes various steps involving one or more hydro-mechanical, thermo-mechanical and extractive chemical procedures to process all parts of the pineapple plant. Referring to FIG. 2, a standard pineapple plant 12 is illustrated. The pineapple plant 12 contains a mother stump 14, a first ratoon stump 16, a butt 1, and a sucker 19. The pineapple fruit 20 rests at or near the highest point of the plant 12. The pineapple fruit 20 contains a crown 22 with crown leaves 24. A slip 26 acts as a rudimentary fruit with an exaggerated crown. The slip 26 develops from buds in the axils of the leaves 28 extending from the fruit stalk or stem 18. Roots 30 help provide the plant 12 needed nutrients and water. The present invention provides for a mechanism to utilize most, in not all of the pineapple plant 12.

Referring back to FIG. 1A, the pineapple biomass disassembling extraction process 10 is designed to process stem homogenate 32, fruit pulp or core 34, fruit peel 36, crown leaves 38, leave homogenate 40, individually or in combination. Each of the components of the plant is further processed to provide additional sub-components. For example, the stem 32 is initially processed to provide an aqueous phase 42 and a solid fiber residue phase 44. Each of the two phases can be further processed to provide for additional isolated components. The processed liquids 42 can be further processed to provide one or more enzymes 46, such as but not limited to SH-esterases I, flour 48, such as functional starch flour containing at least one enzyme, such as bromelain, sugar 50 such as glucose and glucose derivatives, and stem homogenate residuals 52. The solid fiber residue 44 can be processed to provide dietary fiber K-lation fiber 52. Such fibers can form the basis of nutraceuticals 54. The solid fiber residue phase 44 can be further processed to form sugars 56 such as Xylose, Aribinose and other C5 and C6 monomeric sugars, biosurfactants 58, and food additives 60 and cosmoceuticlas 61. The sugars 56 can be processed and/or used for sugar alcohols 56A or low caloric natural sweeteners 56B.

The fruit pulp and core can be processed to provide for fiber juice 62, enzymes such as but not limited to SH-esterases I and other enzyme extracts 64. The enzymes 64 can be used to provide biotherapeutics 66. The fruit peel 36 is further processed to plant hydrolyzates 68, syrups 70, organic acids 72 and biobased chemicals 74.

The fractionated crown leaves 38 can be processed to provide lumen 76, short fibers 80, and micro and nanofibers 80. In addition to the same components processed from the crown leaves, the other leaves of the pineapple plant can be processed to isolate fibers 84 for paper, natural pigments 86, lignin and derivatives 88, and other residuals 90 which may be useful for advanced biofuels.

FIG. 1B provides a generalized overview of the disassembling extraction process 10. The disassembling extraction process 10 starts with the obtaining or provided of plants, such as one or more varieties of pineapples, see step 100. The plants are inspected to insure the plants meet organoleptic and biochemical criteria. The pineapples are washed, step 102. In the washing phase, waste such as dirt and dust is washed away. A second inspection is performed to remove any unwanted raw materials. The plant may be dissected to separate various components, step 104, into for example leaves 106, stems 108, and other components 110, such as fruit body, peels. The leaves 106, stems 108, and other components 110 may be further processed separately. The stems 108 are then fractioned by cutting into predetermined sizes, step 112. The fragmented material can be subject to maceration in water in the blending stage, 114. After maceration, the slurry formed is separated into solids and liquids. The solids are processed to form for example fibers and k-lation. The liquid portion is processed to form for example, flour and bromelain. Various components are separated (i.e. fiber from flour), step 116. Additional separation and centrifugation steps, 120 and 122, are performed. Additional processes are undertaken to extract various plant constituents or products, step 124, such as bromelain, 126, fiber 128, flour 130, or sugars 132.

To the separated leaves 106, several steps are applied thereto to provide additional plant constituents or products. The leaves are preferably processed using a delaminatation step 133 in which layers of plant tissue are removed. For example, the delaminatation process strips away various plant layers, including plant waxes, dermis, epidermis and/or pericarp. The delaminating process forms various components, 1)solubles, which include chlorophyll and nano-fibers, 2) semi solids, which include some nano-fibers, and lumen, and 3) solids, which include long fibers. These leaf first building blocks can be further processed to form additional isolated products. The leaves 106 undergo fraction steps 134, separations steps, 136, and isolation of products 138, such as fiber 138, lumen 140. A paper extraction process 142 occurs via additional steps, such as pre-treatment 144, washing steps 146, bleaching 148, and separation techniques 150.

The pineapple biomass disassembling extraction process includes a minimal step disassembly extraction that efficiently enhances the molecular, plant cellular and tissues separation of the biomass pineapple components. This innovative procedure facilitates the fast separation of the main pineapple plant fibers, biopolymeric constituents and other natural compounds towards their further isolation, purification, refinement and derivatization. The final products can be alternatively separated by specific molecular weights, according to their physicochemical, hydrodynamic, biochemical, mechanical, thermo-mechanical and rheological properties, and/or by sorting them into homogenous materials. By separating the various components of agricultural materials, the pineapple biomass disassembling extraction process 10 offers significant functional extractability, and cost-effective production of fibers and natural compounds, as well as back-end economic and environmental advantages to minimize the land filing of residual agricultural biomass, preventing the production of GHG and other toxic substances, and the elimination of traditional contaminant procedures such as the open field burning. The pineapple biomass disassembling extraction process 10 has many possible applications for both organic and non-organic products.

The pineapple biomass disassembling extraction process is preferably designed as a one step disassembly and extracting method for preparing multiple extractants containing fractions as described above and illustrated in FIG. 1A. The pineapple plant or feedstock comprises natural plant biopolymers listed in Table 1. As shown in the Table 1, the typical pineapple plant includes hemicelluloses, cellulose, lignin as well as starches and multiple active enzymatic systems, constitutive proteins, dietary fiber as well as sugar monomers (glucose, fructose, arabinose, galactose, mannose, xylose) and oligosaccharides sugars (arabinoxylans, glucomannas, arabinogalatans, xyloglucans).

TABLE 1
Analysis of the Main Constituents in the Pineapple Plant

Main Components in
the Residual Pineapple Plant

| | Stems | Leaves |
|---|---|---|
| Constituents | % Dry Weight | % Dry Weight |
| Crude protein | 7.425 | 5.45 |
| Fiber Neutral Detergent | 62.94 | 59.1875 |
| Fiber Acid Detergent | 24.4825 | 31.4375 |
| Cellulose | 17.0425 | 20.6 |
| Hemicellulose | 38.5775 | 27.9325 |
| Lignin | 5.955 | 4.5875 |
| Carbohydrates | 11.775 | 10.325 |
| Chlorophyll | 0 | 16.85 |
| Active Proteins | 1.5075 | 0.3425 |
| Humidity | 12 | 12 |

The pineapple biomass disassembling extraction process results in the production of fibers and several natural products obtained from the pineapple plant such as fibers, enzymes, functional biopolymers and multiple natural compounds. In particular the pineapple biomass disassembling extraction process 10 provides a method of preparing, comprising fractionation, extraction and concentrating the natural products from pineapple plant biomass that comprises the extraction of biopolymeric fibers, dietary fiber, functional starches, proteolytic enzymes as phytotherapeutic agents, α and β-D-Mannopyranosidases, natural pigments, monomeric sugars and low caloric natural sweeteners, comprising; thermo-mechanical maceration and blending of the leaves and stems of the residual pineapple plant. The pineapple biomass utilized includes all components of the pineapple plant and most preferable, the leaves, stems as well as the pineapple peel, crown and center-heart of the fruit.

Various pineapple plant varieties can be used, and include species such as Smooth Cayenne, Red Spanish, Queen and Abacaxi and most preferable pineapple cultivar varieties such as: Hilo, Sugar Loaf, White Sugar Loaf, Kona Sugar Loaf, Natal Queen, Pernambuco, Queen, Green Spanish, Manzana, DelMonte Gold, Perolera, Maipuri, Singapore Spanish, Singapore Canning, Hawaiian Gold, Super Sweet, Ultra Sweet and MD2. Liquid streams, semisolid and solid residues are produced and separated after the pineapple biomass has been subjected to the mechanical disassembling extracting process. Further downstream stages are designed to achieve higher yields and efficiency while maintaining process efficiency using minimal number of stages. Reduction in stages reduces energy, residence time, lowers manufacturing and capital costs. In order to isolate specific fractions according to particle size and consistency various techniques are used, individually or combination, including separation techniques, chromatographic techniques, decanting (decanter centrifuge, hydrocyclone and clarifier, high speed and ultracentrifuge) and filtration systems (ultrafiltration, nanofiltration, reverse osmosis, belt filters, disc and drum filters), including drying support (freeze drier, spray drier, wind tunnel drier). Such system allows for higher levels of separation and purity of the final compounds and products.

The extraction of multiple types of fibers and natural compounds was carried out using several pineapple cultivars such as Smooth Cayenne lisa, Manazana, MD2 and Perolera, widely cultivated in the Central America and South America Regions. Table 2 illustrates several pineapple varieties tested using the process in accordance with the present invention. The table also indicates the presence of several components found in the stems and leaves of each of the varieties.

TABLE 2
Composition of Stems and leaves in varies pineapple varieties:

| Main | | | | | | | | |
|---|---|---|---|---|---|---|---|---|
| Components | | | | | | | | |
| in the Residual | | | | | | | | |
| Pineapple | Cayenne Lisa | | MD2 | | Manazana | | Perolera | |
| Plant | Stems | Leaves | Stems | Leaves | Stems | Leaves | Stems | Leaves |
| Constituents | % Dry Weight | % Dry Weight | % Dry Weight | % Dry Weight | % Dry Weight | % Dry Weight | % Dry Weight | % Dry Weight |
| Crude protein | 7.5 | 5.9 | 8.2 | 6.3 | 7.2 | 4.9 | 6.8 | 4.7 |
| Fiber Neutral | 63.16 | 69.85 | 61.8 | 59.3 | 64.2 | 58.4 | 62.6 | 58.2 |
| Detergent | | | | | | | | |
| Fiber Acid | 24.37 | 31.86 | 23.45 | 32.18 | 24.8 | 30.24 | 25.31 | 31.47 |
| Detergent | | | | | | | | |
| Cellulose | 17.77 | 20 | 16.4 | 21.4 | 16.8 | 19.7 | 17.2 | 21.3 |
| Hemicellulose | 38.79 | 28.99 | 40.15 | 28.51 | 36.22 | 27.31 | 39.15 | 26.92 |
| Lignin | 6.35 | 4.48 | 5.32 | 4.75 | 6.21 | 4.62 | 5.94 | 4.5 |
| Carbohydrates | 12 | 10 | 11.4 | 10.8 | 12.5 | 10.3 | 11.2 | 10.2 |
| Chlorophyll | traces | 17 | traces | 16.8 | traces | 17.4 | traces | 16.2 |
| Active Proteins | 1.5 | 0.2 | 1.7 | 0.5 | 1.53 | 0.4 | 1.3 | 0.27 |
| Humidity | 12 | 12 | 12 | 12 | 12 | 12 | 12 | 12 |

Samples were collected from pineapple growers located within 50 to 70 miles distance from the processing facility. The pineapple biomass was collected from pineapple growers that comply with the characteristics of the biomass required for our process, mainly to keep the freshness of the plants to avoid deterioration and reduction on the concentration of main components that could diminish the extraction yield of the final products.

As part of the process, collected plant material from the field was cleaned in an aqueous media, such as water, to remove soil, fertilizer and other chemical impurities. Preferably, the plant material is collected within 24 hours from the time the plant is picked or harvested for its fruit. The plant biomass was mechanically fractionated, (i.e. via delamination process, abrasion processes, use of water to mix within the tissues and/or starches of the plant, or homogenization process) in order to disassembly the foliage and the rhizome plant components. The pineapple plant varieties utilized for the extractive method were selected in terms of the characteristics of the main parts of the remaining plant such as the stem and leaves to be subjected to the disassembling extractive process. These components were sub-fractionated into small pieces, preferably having a length of 10 mm to 100 mm, preferable 20 mm to 80 mm, and most preferably 25 mm to 50 mm. In the pineapple plant processing, the pineapple biomass was subjected to a disassembly disruptive combination of thermo-mechanical and hydro-mechanical processing that comprises a simultaneous mechanical disruption, plant tissue attrition and homogenization processing, with an attrition abrasion speed between 700 rpm to 5,000 rpm, preferably 850 rpm to 4350 rpm, and most preferable 975 rpm to 3200 rpm for about between 2 to 35. Preferably, the abrasion was carried out for between 7 to min, and most preferably, between 12 to 24 min. The mechanically treated fractionated pineapple biomass feedstock was further subjected into a series of extractive and downstream stages as part of the integrated bio-refining process towards the production of various types of biofibers, several natural products and derivatives.

Stem Processing

Production of Bromelain/Enzymes: The rhizome stem fraction of the plant biomass was further extracted in order to enhance the solubility of the target bromelain protein system (E.C.3.4.33.4) and pineapple thiol-endopeptidases. The processing of the stems as well as pineapple core and peel required an equilibration into a solution of 1.5% to 3.7% of NaCl, more preferable 2.5% to 3.0%, most preferable 2.0% to 2.7% for about 120 minutes to about 210 minutes, most preferably 150 minutes. The material was subjected to extractive homogenization in the presence of sodium acetate buffer 0.01M at a working pH 5.7 to 7.2, more preferably pH 5.2 to 7.0, and most preferably pH 5.5 to 6.8. The extractive homogenization proceeded at 5,000 to 45,000 rpm, more preferably 8,000 rpm to 35,000 rpm and most preferably 12,000 rpm to 28,000 rpm in a temperature range from 5° C. up to 15° C. The homogenization process took place for a period of at least 70 seconds, and up to about 45 minutes.

Following the mechanical homogenization process, a filtration separation stage was performed to further isolate the bromelain (cystein-endopeptidases) crude extract. The filtration process resulted in obtaining an aqueous filtrate and a solid residue. The aqueous filtrate was further decanted at a temperature of 5° C. to 9° C., more preferably at 4° C. to 7° C., most preferable at 3° C. to 5° C. A clear liquid phase was separated from the soft semisolid residue and the liquid phase was further centrifuged at 1600 g for 25 minutes, more preferably at 2700 g for 10 minutes, and most preferable at 2100 g per 20 minutes. The clarified liquid fraction was further characterized by determining the protein content using techniques known to one of skill in the art, such as the Bradford method and enzyme activity. After the centrifugation process, the semisolid precipitant was concentrated and comprised a biopolymeric mixture of 2% pectins, 5% hemicelluloses and 97% starchy type polymers. Usually the starch fraction is stored in the stem of the plant instead of the fruit. Prior to fruit ripening, the starch reserves are enzymatically converted to monomeric sugars and further accumulated in the fruit, thereby increasing the sweetness.

The aqueous phase that contains the bromelain enzyme system was subjected to a fractional precipitation stage comparing both ammonium sulfate and ethanol as complementary step. Prior to that, the pH of crude enzyme aqueous solution was adjusted to a pH 5.3 to pH 8.2, more preferably between pH 5.7 to pH 7.5, most preferably between pH 6.2 to pH 7.0. The initial fractional precipitation was carried out by combining the addition of ammonium sulfate to achieve different percentages of saturation, ranging from, for example, 20% to 95%, more preferably 35% to 50% and most preferably 41% to 47%. The fractional precipitation was carried out at a temperature between 2° C. to 7° C.

Ammonium sulfate was added to achieve saturation under constant stirring using a magnetic stirrer. After adding the ammonium sulfate, stirring was continued for 15 more minutes to allow attainment of equilibrium between the dissolved and aggregated protein. The salt enriched solution was then subjected to high speed centrifugation at 1800 g for 25 minutes at 4° C. The precipitate pellet was collected and resuspended in 0.01M Phosphate buffer pH 7.2. The clear supernatant was recollected and the volume was utilized for further saturation with ammonium sulfate. Another fraction of the crude soluble extract was precipitated stepwise by mixing with different concentrations of cold ethanol (v/v) until a percentage of 20% to 85%, more preferable 30% to 65% and most preferable 40% to 75%, was achieved. The final ethanol enzyme crude extract solution was centrifuged at 1800 g per 25min at 4° C. The protein pellet was resuspended in 0.01M Phosphate buffer pH 7.2.

As depicted in Table 3, the fractional precipitation with Ammonium sulfate or ethanol was both efficacious alternatives to precipitate the bromelain enzyme system. It is possible to achieve up to 27% yield with the above precipitation tools. Alternative to, or in addition to the selective centrifugation process, microfiltration, ultrafiltration and lyophilization or other drying processes may be utilized. The use of fractional precipitation with Ammonium sulfate and ethanol appears to concentrate the target enzyme products, thereby minimizing the crude enzyme working volume to facilitate further downstream operations. Such action optimizes time and cost savings in order to reduce the purification and downstream stages of the bromelain enzyme system process.

TABLE 3
Fractional Precipitation of Bromelain with Ammonium sulfate and Ethanol:

| | Protein | | | | |
|---|---|---|---|---|---|
| | Concentration | Bromelain | Specific Activity | | Purification |
| | (mg/ml) | Activity (U/ml) | (U/mg protein) | Yield % | Factor |
| Fractional Precipitation | | | | | |
| with Ammonium Sulfate | | | | | |
| Bromeline Crude Extract | 0.345 | 31.98 | 92.7 | — | — |
| 0 to 25% | 0.003 | 0.0927 | 30.9 | 0.8 | 0.33 |
| 25% to 45% | 0.095 | 41.7 | 438.94 | 27.53 | 4.73 |
| 45% to 65% | 0.062 | 6.52 | 105.16 | 17.97 | 1.13 |
| 65% to 75% | 0.044 | 4.31 | 97.95 | 12.75 | 1.05 |
| 75% to 95% | 0.01 | 0.3 | 30 | 2.8 | 0.32 |
| Bromeline Precipitation | | | | | |
| with Ethanol | | | | | |
| Bromeline Crude Extract | 0.345 | 31.98 | 92.7 | — | — |
| 0 to 20% | 0.005 | 0.041 | 8.2 | 1.44 | 0.0088 |
| 20% to 65% | 0.09 | 28.36 | 315.11 | 26.08 | 3.39 |
| 65% to 85% | 0.035 | 5.24 | 149.7 | 10 | 1.61 |

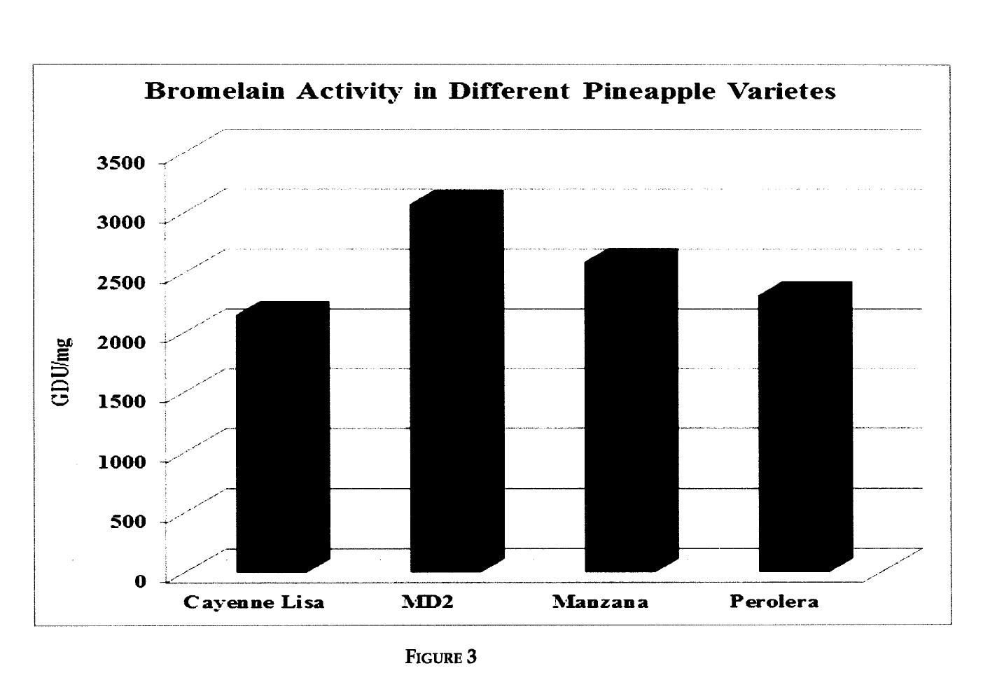

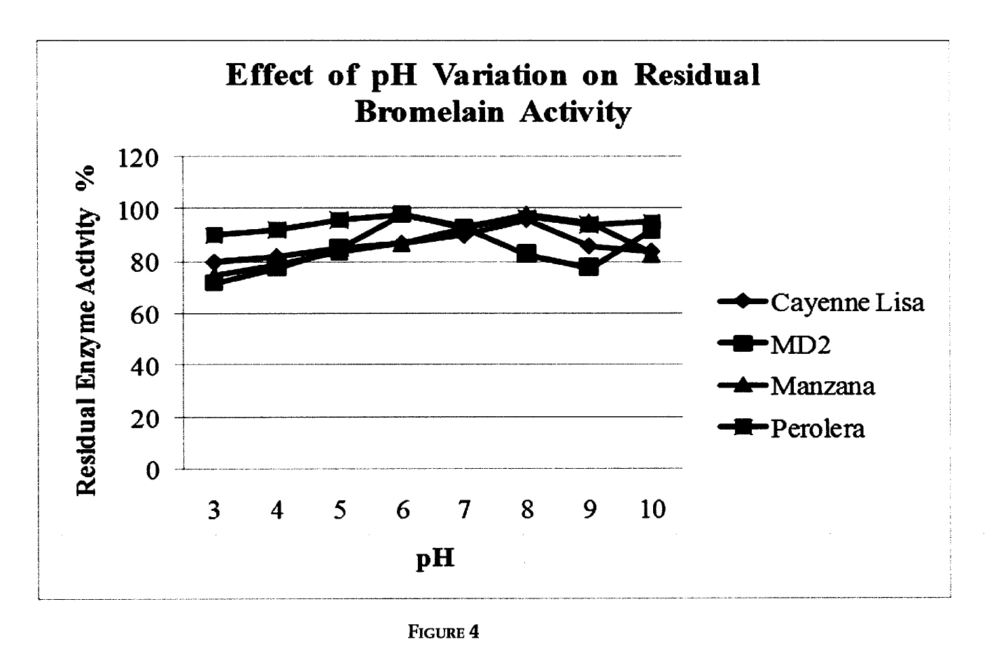

The activity content of bromelain among different varieties of pineapple was indicated at FIG. 3. As indicated the MD2 exhibited the highest bromelain activity 3250 GDU/mg, followed by the varieties manzana and cayenne lisa and perolera. FIG. 4 further indicates the pH variation effect on the residual Bromelain activity, illustrating the range of enzymatic activity at various pH ranges.

STEMS: FLOUR STARCH/FIBER: After mechanical pretreatment of the pineapple biomass, the liquid fraction and the solid fiber residue were separated for further extraction. The liquid fraction was further decanted at a temperature range of about 0° C. to about 17° C., preferably about 4° C. to about 12° C., more preferably from about 2° C. to about 9° C., to obtain a precipitant. The precipitant contained a mixture of insoluble biopolymers. After the decanting process was concluded, the supernatant was removed from the precipitant material. The precipitant fraction was constituted mainly of natural materials such as hemicelluloses, pectin and complex starch.

TABLE 3
Composition and/or Characteristics of the isolated Flour.
Pineapple Stem Flour Composition

| COMPONENT | % |
|---|---|
| Hemicellulose | 8.4 |
| Cellulose | 12.3 |
| Pectin | 2.8 |
| Starch | 45.7 |
| Protein | 6.2 |
| Glucose | 2.8 |
| Xylose | 2.1 |
| Xyloglucans | 1.7 |
| Sucrose | 1.9 |
| Fructose | 2.1 |
| Lipids | 2.8 |
| Water Content | 7.1 |

These natural materials can be isolated and processed further according to specific industrial applications. The crude residual fraction after homogenization of the stems was mixed with water in a proportion of 2:1. Alternatively, the proportion can be 2.5:1 or 3:1.5. The homogeneous aqueous phase was isolated after the decantation stage. The pH on this working solution was adjusted at a pH of between 4.5 and 7.5, preferably 4.7 to 7.2, most preferably from 5.5 to 6.8. Once adjusted, fractional precipitation was performed by adding solid ammonium sulfate to saturation between 45% to 79% at cold temperature. After standing for a couple of hours, the precipitate obtained contained active crude protein fraction, mainly sulfhydryl cysteine proteinases (EC 3.4.22.32) comprising a mixture of more than five isoforms. The five isoforms exhibited multiple post-translational modifications. The extract also contained other accessory enzymes having activities such as peroxidases, cellulases, xylanases, phosphatases, acetylxylanesterases, β-D-galactosidases, β-D-N-Acetylglucosaminidases, pectinases, α-D-Mannosidases and β-D-Mannopyranosidases. The presence of cystein protease and glycosidase activities in the bromelain mix indicated that a cleavage effect on the carbohydrate glycosylic fraction of multiple proteins besides the proteolitic activity, thereby expanding the radius of industrial, medical and pharmaceutical applications of the bromelain extract.

Dietary Fiber Applications: The delignified solid residue was a fibrous material with a high content of cellulose. The material was further dried and milled to produce nanofibers that can be incorporated into dietary fiber. Such dietary fiber can be used for food consumption incorporated into multiple food ingredient applications. In the embodiment of the present invention, the solid plant material of the pineapple biomass was subjected to mechanical disassembling disruption and homogenization process, exerting processing attrition conditions for mechanical disruption and homogenization effects. Such processes are performed in combination with abrasion and maceration during this edge-to-surface stage. A mechanical treatment and friction between fibers provided fiber-to-fiber treatment and vortexed effect at higher rotation speed between 700 rpm to 5,000 rpm, more preferable 850 rpm to 4350 rpm and most preferable 975 rpm to 3200 rpm for about 2 to 35 minutes, more preferable 7 to 28 minutes and more preferable 12 to 24 minutes, in order to detach pericarp tissue.

The process enables concentrating significant higher levels of biofiber of different lengths “Long fibers” and “Short Fibers” as well as “micro fibers” and “nano fibers” respectively, as well as several compounds distributed within the water soluble and insoluble fractions. Separation of the soluble from the solid residual fractions was performed through the filtrating stage which involves a temperature in the range of from 3° C. to 10° C., for a period of 2 hours and preferable from 2 hours to 24 hours preferable at a temperature range of 2° C. to 4° C. The present method may include further separation, nanofiltration, microfiltration and ultrafiltration steps, including additional chromatographic techniques such as gel filtration, hydrophobic interaction, anion exchange, cation exchange, affinity chromatography steps for the isolation and polishing to homogeneity multiple natural proteins, peptides and enzymes, as well as monomeric sugars, oligosaccharides, biopolymers, pigments and complex carbohydrates and fibers from each of the produced fraction

LUMEN: During the disassembly of the pineapple plant biomass, another fibrous fraction was produced. A fraction with more short length fibers and pericarp tissue fragments was isolated. This product is the material once the long fibers are removed. The lumen portion may therefore contain pieces of short fiber, leaves, epidermal tissue and chlorophyll. This wet fraction was highly regarded for its cellulose content and other plant components. The reformulation of this lumen fraction can be amenable for the production of cattle feed, aquaculture, pet food and other food and nutritional related applications. Further extractive operations on this lumen fraction such as delignification and bleaching can be done to incorporate the refined fibers into paper manufacturing or higher value applications.

CHLOROPHYLL: An additional component isolated from the mechanical extraction process undertaken by the pineapple biomass plant was natural pigment Chlorophyll. This organic compound can be obtained in enough levels as a by-product and further concentrated into either liquid or powder form. A primary use for The Chlorophyll includes industrial applications as natural pigments in substitution of petroleum derived pigments. Chlorophyll may also be used in food applications, pharmaceutical applications, as well as the medical and diagnostics applications.

Extraction Process Fiber K-Lation: Fiber K-Lation can be obtained from isolating the crude residue after the extractive homogenization of the pineapple biomass rhizome stems. The product is subject to a steam pretreatment, then milled and dried under mild air conditions. The utilization of steam regulated conditions helps to strip certain sugars, complex gums and pectin derivatives to improve the concentration of active and functional ingredients in the product. The product contains active and functional components: a mixture of endogenous constitutive natural plant enzymes such as cystein-proteinases, cellulases and xylanases in combination with soluble and insoluble dietary fiber. The plant enzymes and the soluble and insoluble dietary fiber may be mixed with another fiber source from the plant Trichantera gigantean. The proximal composition of the K-Lation product is indicated in Table 5, where ratios are given based on 100 g of product and amino acid content, Table 6. The K-Lation product has use for multiple applications, including as a dietary supplement to trap cholesterol, accelerate fat degradation and improve function of the GI tract.

TABLE 5
Nutrient Composition of the K-Lation Product

| Chemical Composition | mg/g |
|---|---|
| Protein | 0.176 |
| Total Fiber | 0.231 |
| Lignin | 0.064 |
| Hemicellulose | 0.22 |
| Ash | 0.023 |
| Calcium | 0.061 |
| Phosphates | 0.003 |
| Magnesium | 0.01 |
| Potassium | 0.029 |
| Sodium | 0.006 |
| Sulphur | 0.004 |
| Selenium | 0.0004 |
| Copper | 0.00015 |
| Zinc | 0.00047 |
| Magnesium | 0.00018 |
| Iron | 0.00019 |
| Bromelain | 0.005 |

TABLE 6
Content of Essential and Non-Essential Amino
Acids in K-Lation Product:

| | mg/g |
|---|---|
| Essential Amino Acids Composition | |
| ARGININE | 0.0105 |
| HISTIDINE | 0.004 |
| ISOLEUCINE | 0.0083 |
| LEUCINE | 0.0149 |
| LISINA | 0.0086 |
| PHENYALANINE | 0.0098 |
| TREONINE | 0.0086 |
| VALINE | 0.0103 |
| Non-Essential Amino Acids | |
| ALANINE | 0.0099 |
| AC. ASPARTICO | 0.0171 |
| ACIDO GLUTAMICO | 0.0203 |
| GLICINE | 0.0103 |
| PROLINE | 0.0085 |
| SERINE | 0.008 |
| TYROSINE | 0.007 |

As part of the preparation of the K-Lation mixture, the solid residue or fiber was washed with hot water three (3) times to eliminate the sugar content. Immediately following the washing, a second product, Trichantera Gigantea, was added to enable fat burning action. After mixing of the ingredients, a fractionating process was carried out. The fractionating process was carried simultaneously during a grinding process. Bulk fiber was obtained with the particle diameter selected in accordance to the characterization of a dietary supplement, regulated by INVIMA (FDA). The product was subjected to direct heating to eliminate water content until reaching 8-10% humidity. The selected fiber may be encapsulated in capsules or further processed for oral use.

Xylose Extraction: The crude residual fraction after the extractive homogenization stage was mixed with water in a proportion 2:1(v/w) and diluted with sulfuric acid (0.7%). The mix was heated under recirculation for about 30 minutes to 200 minutes. The heated mixture was filtrated to obtain a liquid phase and a solid residue. As a result of the acid hydrolysis process upon the hemicelluloses fiber component on the crude residual fraction, the liquid fraction contained a higher concentration of Xylose sugar as well as other Xylose derivatives. The sugar was further concentrated and refined, using for example additional processes such as evaporation, dewatering, or centrifugation, to obtain a product ready for several food, nutritional and preparation of low caloric sweeteners. At present the regiospecific hydrolysis of arabinoxylans in pineapple biomass becomes an important requirement to enhance the utilization of pineapple hemicelluloses and heterosubstituted arabinoxylans, glucomannas, arabinogalatans, xyloglucans in the advance liquid biofuels sector, food, pharmaceutical, low caloric nutritional products and so forth.

An alternative mechanism to produce monomeric sugars included utilization of specific hydrolytic enzyme systems that have the capacity to produce xylose and complex heterosubstituted xylose derivatives that in mild reaction conditions and high speed reaction rates, becomes a moderate and less aggressive way to produce higher yields of xylose and xylose derivatives than the use of sulfuric acid. Therefore in the embodiment of the present invention the enzymatic hydrolysis of hemicelluloses pineapple biopolymers allowed for production of significant yields of xylose in a more sustainable way than the use of sulfuric acid. For the isolation of xylose and xylose derivatives, it is necessary to carry out starch and protein removal.

Pineapple Stem Fiber suspension 100 g: Stem fiber was treated with wheat bran (2 uL/g wheat bran) at a temperature range of 80° C. to 90° C., more preferable 83° C. to 85° C. and most preferable at the range 82° C. to about 87° C. per about 60 minutes. The mixture was filtered through bolting cloth, re-suspended and repeatedly treated with Thermozyme for 90 minutes, more preferable 80 minutes, most preferable 120 min. The suspension was filtered, washed with water and Tris-HCl buffer (0.1 M, pH 7.8). The obtained destarched PSF was resuspended in Tris-HCl buffer and treated with Protolichetherm protease (20 mg/g destarched stem fiber, 45° C., 20 hours) followed by filtration. Pretreated PSF solids were resuspended in water and boiled for 20-30 minutes, at a temperature of between 85° C. and 100° C. for enzyme inactivation. The slurry was washed and filtered 5 to 7 times with deionized (DI) water and air-dried at 65° C. for 10 hours to 18 hours. As a result, 23.8 g of destarched and deproteinized stem fiber was obtained.

Preparation of xylose and xylose derivatives from pineapple stems fiber as follows: destarched and deproteinized stem fiber was suspended in NaOAc buffer (0.1 M) at a pH 5.0, more preferable pH 5.3, most preferable pH 5.7 to give 100 g/L suspension. Xylanolytic, Cellulolytic, Pectinolytic Enzymes were added on the following dosage ranges 0.4 U/g to 15 U/g DD-stem fiber and incubated at 50° C. to 60° C. Samples were subtracted after 24, 65, and 110 hours of hydrolysis, centrifuged, and subjected to HPLC analysis. A chromatographic method that uses HPAEC-PAD High Pressure Anio Exchange Chromatography with Pulse Amperometric Detection was developed as follows. The DD-PSF samples after hydrolysis were centrifuged at 11,000 g 16,000 g, more preferable 14000 g, most preferable 16,000 g per 10 to 15 minutes, heated at 85-100° C., for 10 minutes. After dilution in deionized water in the presence of 0.053% to 0.75% NaN3, samples were subjected to HPLC analysis. The analysis was performed with an Agilent 1100 HPLC system with Esa Coulochem III electrochemical detector and Dionex CarboPac PA-100 column (HPAEC-PAD).

Xylose carbohydrates and xylose derivatives were separated by anion exchange chromatography HPAEC-PAD at high pH and detected by pulsed electrochemical detection. Electrochemical detection was used to measure the current resulting from oxidation or reduction of analyte molecules at the surface of a working gold electrode. The chromatographic running conditions used were: Column: CarboPac PA100 (4 x 200 mm) and Guard (4 x 50 mm), Eluent: A: 50 mM, preferable 100 mM, most preferable 175mM NaOH; B: 250 mM, preferable 370 mM, most preferable 400mM of NaOAc in 65 mM, more preferable 75mM, more preferable 100mM of NaOH Gradient 0-62.5% B in 30 minutes, 62.5-100% in 15 min, followed by 75mM, more preferable 80mM, more preferable 100mM NaOH for reconditioning for 12 minutes to regenerate the column. Flow Rate: 0.75mL/min, more preferable 1.00 mL/min, most preferable 1.5 mL/min. Temperature: ambient Detection: Pulsed electrochemical, Au electrode. Waveform: Quadruple potential, Arabinose, xylose, xylobiose, xylotriose, and xylotetraose were used as standards.

Enzymatic combinations were diluted in 50mM NaOAc buffer pH 5.7, more preferable pH 6.3 and most preferable pH 7.0 to screen multiple enzyme dosages ranges such as 0.4 U/g to 0.7 U/g; more preferable 2 U/g to 4 U/g, most preferable 10 U/g to 15 U/g DD-PSF in the hydrolysis mixture and incubated at 50° C., more preferable at 54° C. and most preferable at 57° C. Samples were subtracted after 24, 65, and 110 hours of hydrolysis. Calibration of anion-exchange column was performed using xylose and xylooligosaccharides with DP 2-4. The coefficients for calculation of peak area to concentration were approximated using extrapolation of the calibration plot.

Xylitol: The Xylose obtained in the methods as described above was subjected to alternative chemical hydrogenation under the following working conditions using hydrogen gas under the presence of a chemical catalyst. As an illustrative example, Nickel was used as the chemical catalyst under the working conditions of 10 atmosphere of pressure and 55° C. to 95° C., more preferable 65° C. to 90° C., most preferable 70° C. to 85° C. during a process time of around 30 minutes to 200 minutes, more preferable 45 minutes to 180 minutes, most preferable 60 minutes to 120 minutes.

LIGNIN: The first solid residue obtained after the extractive maceration of the pineapple biomass was subjected to a treatment with 1% to 3.5% NaOH, more preferable 1.5% to 3.0%, and most preferable 1.8 to 2.5% Na0H, in a proportion 2:1 (volume to weight). The mixture was heated between 55° C. to 95° C., more preferable 65° C. to 90° C., most preferable 70° C. to 85° C. during a process time of around 30 minutes to 200 minutes, more preferable 45 minutes to 180 minutes, most preferable 60 minutes to 120 minutes. At the end of the heating step, the mixture was filtrated, the fibrous solid residue was separated with the liquid fraction containing mainly polymers of lignin. Further concentration of this material can be utilized for the production of flavor (vanillin) and other food ingredients.

Leaves: The embodiment of the present technology invention involves an innovative method of one step disassembly single and integrated extraction of pineapple biofibers in which the pineapple biomass was subjected to mechanical disassembling disruption and homogenization process, exerting processing attrition conditions to facilitate the mechanical disruption of the plant tissue. In the embodiment of the present invention multiple combined effects were exerted to efficiently increase the disassembly disrupting effects through and mainly external and internal defibrillation, delamination and multiple microcompressions on the pineapple biomass plant cell walls. In the embodiment of the present invention the simultaneous disassembling disruptive effects, most preferable in combination with abrasion, delamination and maceration effects performed during this edge-to-surface stage providing a hydro-mechanical effect and maintaining the extraction environment at pH range of 4.9 to 7.5, more preferable pH 5.8 to 6.2, and most preferable at a pH range of 6.7 to 7.3 and a fiber extraction temperature of 15° C. to 25° C., more preferable at the range of 17° C. to 27° C. and most preferable at a temperature range of 12° C. to 21° C. In the embodiment of the present invention the process induces natural surface active friction and abrasion between fibers gives enabling fiber-to-fiber treatment. In the embodiment of the present invention our unique integrated process helps to enhance favorable biostructural fiber arrangements and molecular redistribution from the inner areas of the fiber bundles to the exterior, including the release and exposure of multiple natural biopolymers and complex carbohydrates within the plant leaves matrix, such as: pectin, hemicelluloses, cellulose, some lignin species, protein-sugar and protein-oligosaccharides colloidal materials, the overall process improve the interfiber surface area interaction which enhances the reactivity of the fibers for further industrial processing and applications.

The pineapple biomass disassembling extraction process enhances the molecular, plant cellular and efficient plant tissues separation of the biomass pineapple leaf components. The plant biomass was mechanically fractionated in combination with the delamination of the foliage the pineapple leaves and further subjected to a disassembly disruptive combination of thermo-mechanical and hydro-mechanical processing that comprises a simultaneous mechanical disruption, plant tissue attrition and maceration processing, with an attrition abrasion speed between 700 rpm to 5,000 rpm, more preferable 850 rpm to 4350 rpm and most preferable 975 rpm to 3200 rpm for about 2 to 35 min, more preferable 7 to 28 min and more preferable 12 to 24 min, to efficiently detach components of the pericarp tissue. At this stage the fibers aggregates and are not free to move independently. Instead they form flocs of 2mm to 7mm, and under the continuous shear, delaminating and turbulence hydrodynamic forces, some bundles of fibers may be further defibrillated. In the embodiment of the present invention the mechanically treated fractionated pineapple biomass was further subjected into a series of extractive and downstream stages as part of the integrated biorefining process towards the production of various types of biofibers, several natural products and derivatives. In the embodiment of the present invention from the fibrous material which was subjected to the disassembling fractionation process, three streams were produced that corresponded mainly to the long fibers, Lumen (short fibers) with fines and water soluble residuals. The process enabled concentrating significant higher levels of biofiber of different lengths long, short, micro nano fibers respectively, as well as several compounds distributed within the water soluble and insoluble fractions. The present method may include further separation, nanofiltration, microfiltration and ultrafiltration steps, including additional chromatographic techniques such as gel filtration, hydrophobic interaction, anion exchange, cation exchange, affinity chromatography steps for the isolation and polishing to homogeneity multiple natural proteins, peptides and enzymes, as well as monomeric sugars, oligosaccharides, biopolymers, pigments and complex carbohydrates and fibers from each of the produced fractions.

During the disassembling of the plant biomass it was possible to develop a positive refining effect to the pineapple natural fibers. In some regions of the fibers this refining effect brought several biostructural fiber enhancement effects. The fiber walls were to some extent removed through external fibrillation, or “rooting out” and delamination effects which creates fines and debris in suspension in the form of micro (1 μm to 3 μm) sizes, such as elementary fiber and nano fibers (3 nm to 12 nm). Internal biostructural changes occurred in the cell walls such as swelling and internal fibrillation.

In an embodiment of the present invention, it was possible to enhance fiber mechanical characteristics as indicated in TABLE 7. Table 7 depicts the mechanical characteristics of pineapple leaf fibers from various pineapple cultivars. Some of the differences on the mechanical characteristics among the fibers from each pineapple variety were attributable to the differences in fiber conformation, orientation, internal and external bio-structural rearrangements occurring during the extracting disassembling process. In addition the possible reactivity among hydroxyl groups along the fiber may enhance the interfacial fiber-fiber interaction to form strong bundles and microfiber to nanofiber aggregates. Exogenous forces, such as stressing, deforming, elongating, tensile, impact strength and flexural, may induce morphological and biostructural changes. The strength and nature composition of the pineapple fibers, with more than 82% cellulose, results in a relatively stable polymer, with the fiber-to-fiber and interfacial adhesion in the fiber bundles attributing to the density on the hydrogen bond network. As a result, the pineapple cellulose fibers have a good flexibility and elasticity, allowing for maintaining high processing characteristics. These improved mechanical properties may enhance the properties of the materials where pineapple fiber are incorporated, for instance in the papers making process, biocomposites, biopolymers of multiple fiber industrial applications.

TABLE NO. 7
Mechanical Properties Natural Fibers from
Multiple Pineapple Varieties.
Mechanical Properties of Pineapple Natural
Fibers from Different Pineapple Cultivars

| | Pineapple Cultivars | | | |
|---|---|---|---|---|
| Fiber Properties | Cayenne Lisa | MD2 | Manzana | Perolera |
| Diameter (μm) | 30-45 | 24-45 | 52-74 | 38-57 |
| Density (g/cm3) | 1.53 | 1.38 | 1.46 | 1.42 |
| Young's Modulus (GPa) | 51.72 | 45.36 | 38.12 | 42.65 |
| Tensile Strenght (MPa) | 853 | 745 | 572 | 691 |
| Elongation Breaks (%) | 1.6 | 2.1 | 1.5 | 1.8 |

The mechanical properties displayed in Table No. 7, help to better apply pineapple fibers according to the intrinsic mechanical properties to better maximize industrial and commercial value of the pineapple fibers. Every pineapple cultivar from which fibers were extracted displayed diverse mechanical properties. These mechanical properties may increase during fiber processing to increase the internal bonding strength to maximize, drainage resistance, the stiffness, burst and tensile strength. The parameters indicated the tensile strength was higher for the Cayenne Lisa variety followed by the Perolera, MD2 and Manzana. Cayenne Lisa exhibited higher Young's Modulus and density. The mechanical properties that pineapple fibers exhibited helped to further develop structural combinations with other biopolymers and resins which bring new mechanical properties for further industrial applications.

After the process disassembling and extractive stage, the main fiber streams such as long fibers and short fibers or lumen were obtained. Table No.2 indicates the chemical composition of pineapple fibers from various cultivars showing the percentage of cellulose, hemicelluloses, lignin and moisture content. Preferably, the fibers obtained as result of the extractive disassembling process were enhanced. For example, the long fibers exhibited fiber content from 83.11% to 87.16%.

TABLE NO. 8
Chemical Composition of Pineapple Fibers Before and After Bleaching

| | Cayenne Lisa | | | MD2 | | |
|---|---|---|---|---|---|---|
| Leaf Fiber Fractions | Dried Fiber | Lumen | Bleached Fiber | Dried Fiber | Lumen | Bleached Fiber |
| Leaf Fiber Components | | | | | | |
| Cellulose (%) | 85.36 ± 1.47 | 82.4 ± 2.16 | 97.25 ± 1.72 | 87.16 ± 1.36 | 81.31 ± 2.18 | 98.24 ± 1.83 |
| Hemicellulose (%) | 12.15 ± 2.51 | 4.31 ± 2.11 | 1.31 ± 2.11 | 11.95 ± 1.83 | 3.71 ± 2.16 | 1.14 ± 1.97 |
| Lignin (%) | 2.98 ± 1.05 | 1.86 ± 1.10 | 0.62 ± 1.07 | 2.93 ± 1.25 | 1.72 ± 1.12 | 0.84 ± 1.21 |
| Moisture Content (%) | 12.98 ± 1.05 | 12.85 ± 1.25 | 12.98 ± 1.05 | 12.35 ± 1.05 | 12.41 ± 1.12 | 12.45 ± 1.20 |
| | Manzana | | | Perolera | | |
| Leaf Fiber Fractions | Dried Fiber | Lumen | Bleached Fiber | Dried Fiber | Lumen | Bleached Fiber |
| Leaf Fiber Components | | | | | | |
| Cellulose (%) | 83.11 ± 1.26 | 80.71 ± 2.14 | 96.34 ± 1.62 | 86.53 ± 1.80 | 82.16 ± 2.16 | 95.64 ± 1.22 |
| Hemicellulose (%) | 12.38 ± 1.61 | 5.31 ± 2.74 | 2.05 ± 1.85 | 11.94 ± 1.21 | 3.81 ± 2.41 | 1.95 ± 1.35 |
| Lignin (%) | 2.33 ± 1.85 | 1.54 ± 1.16 | 0.80 ± 1.46 | 2.58 ± 1.46 | 1.35 ± 1.63 | 0.86 ± 1.79 |
| Moisture Content (%) | 11.96 ± 1.05 | 12.11 ± 1.17 | 12.05 ± 1.42 | 12.08 ± 1.51 | 12.29 ± 1.39 | 12.72 ± 1.42 |

Bleaching treatment was necessary to remove lignin, lignin derivatives and residual hemicelluloses. The bleaching process further enhanced the mechanical properties of the fibers. The pineapple biomass disassembling extraction process 10 further results in increase strength of the pineapple fibers. These can be further processed through bleaching or biobleaching process conditions with the alternative use of a mix of hydrogen peroxide 0.1% to 0.42%, more preferable 0.15% to 0.35%, and most preferable 0.37% to 0.5% in combination with sodium hypochlorite in a concentration of 3% to and 6%, more preferable 2.8% to 4.5%, most preferable 3.8% to 5.2% respectively. After the extraction of the long fibers, additional lignin and hemicelluloses were removed. As indicated in the levels of cellulose content after the bleaching stage were from 95.74% in Perolera bleached fibers to a maximum of 98.24% obtained with MD2 bleached fibers respectively.

Pineapple Natural Fibers in Pulp and Paper Utilization. The pineapple biomass disassembling extraction process 10 was used to prepare long fibers of pineapple plant, having fibers that exhibit unique mechanical properties, higher cellulose composition, and orientation. The pineapple biomass disassembling extraction process 10 confers excellent surface fiber surface treatment to enhance the mechanical properties of the fibers that are also transferred to the materials where these fibers are incorporated. The pineapple biomass disassembling extraction process 10 may also be adapted to produce both long and short fibers source multiple types of materials and mixture composites, to increase the weightless, mechanical resistance and enhanced optical properties of multiple types of paper and packaging products, light and resistant materials and so forth, but most important the key function of the pineapple fibers is to bring strength, weightless to the final products. More natural fibers are required to substitute chemically recycled fibers which have already been processed and carrying higher concentration of chemicals. In the embodiment of the present invention the process becomes a source of natural fibers to substitute chemically recycle fibers for paper, food packaging, textiles and multiple other applications. The combinatorial extracting disassembling process helps to increase fiber width that amplifies the surface contact areas to increase the fiber bond. This affects favorably on paper properties. Using the pineapple biomass disassembling extraction process 10, the fiber coarseness decreases after the extracting processing. The decrease of coarseness increases the fiber surface and increases the bonding ability of fibers. This property supports an increase in paper strength, and paper smoothness. Fiber width increase means the contact surface of fiber is wider and larger then it increases the bonding between fibers. The higher the fiber width approached, the higher the tearing resistance. Fiber width increase their width may because of flattening of fiber coarseness, and collapse ability. The incorporation of pineapple fibers in the paper mix brings superior mechanical characteristics that also determine the quality and industrial applications of the final product.

The pineapple fibers generated by pineapple biomass disassembling extraction process 10 can be utilized individually or in combination with recycled fibers. The pineapple fibers created have a natural white color that allows production of higher quality papers. Paper formed could be for example, art paper and other fine paper applications that exhibit strong and smooth paper sheet with good printing properties. The long pineapple fibers (LPF) obtained have an average of 1.5 cm to 3.5 cm length. Traditional fibers have smaller lengths, averaging lengths of 0.9 cm to 1.3 cm. The mechanical properties of the LPF obtained bring strength, flexibility and great appearance to paper, such as brightness, opacity, resistance, and long shell life. Samples of paper were made using fibers obtained form the pineapple biomass disassembling extraction process 10.

In addition, the use of combined long and short pineapple fibers, fibers obtained from the pineapple biomass disassembling extraction process 10 can be incorporated to make a good sheet formation or to develop other pulp properties such as absorbency, porosity, brightness or optical properties specifically for a given paper grade. In an embodiment of the present invention, paper samples were prepared according to the TAPPI standards in which fiber concentrations were varied 25% to 35%, second case 45% to 50%, third case 65% to 75% and fourth case 85% to 97%, as indicated in Table 9. Table 9 indicates various optical properties of the paper samples with increasing fibers being incorporated in the pulp mix.

TABLE NO. 9
Characterization of Paper made with Natural Pineapple Fibers
CHARACTERIZATION OF PAPER MADE
WITH PINEAPPLE FIBER BY TAPPI METHODS

| | SAMPLE 1 | SAMPLE 2 | SAMPLE 3 | SAMPLE 4 |
|---|---|---|---|---|
| BRIGHTNESS | 43.15 | 44.55 | 63.12 | 63.54 |
| R(X) | 59.78 | 60.78 | 79.58 | 77.95 |
| R(Y) | 56.16 | 57.16 | 76.39 | 75.18 |
| R(Z) | 42.34 | 43.74 | 62.04 | 62.66 |
| L | 74.94 | 75.61 | 87.4 | 86.71 |
| a | 0.25 | 0.42 | −0.68 | −0.63 |
| b | 12.91 | 12.43 | 11.49 | 10.72 |
| L* | 79.71 | 80.27 | 90.04 | 89.48 |
| a* | 0.26 | 0.45 | −0.67 | −0.63 |
| b* | 14.83 | 14.17 | 12.25 | 10.72 |
| L* | 79.71 | 80.27 | 90.04 | 89.48 |
| C* | 14.83 | 14.18 | 12.27 | 10.74 |
| h* | 88.98 | 88.19 | 93.14 | 93.34 |
| ASTM WH | 0.88 | 3.46 | 18.99 | 25.07 |
| ASTM YEL | 31.05 | 29.81 | 22.97 | 20.35 |
| HUNT YEL | 24.61 | 23.49 | 18.78 | 16.66 |
| CIE WH | −23.36 | −18.36 | 17.01 | 22.96 |
| CIE TINT | −11.17 | −11.04 | −6.59 | −5.73 |
| OUTSIDE | | | | |
| WHITE | | | | |
| LIMITS | | | | |

For example, the levels of brightness increased by increasing the concentration of natural pineapple fibers extracted by the present invention. As shown in the table, brightness levels reached up to 63.54, which after additional washes, but without bleaching reached, was increased to 80. In some other cases, if the concentration of fibers exceed optimal concentration, then the brightness, opacity, whiteness of hand sheet paper may decreased because of increase the density of fiber, fiber contact areas, fiber width, coarseness, levels of hemicelluloses and lignin being released. The paper made with pineapple fibers can be recycled between 3 to 7 times.

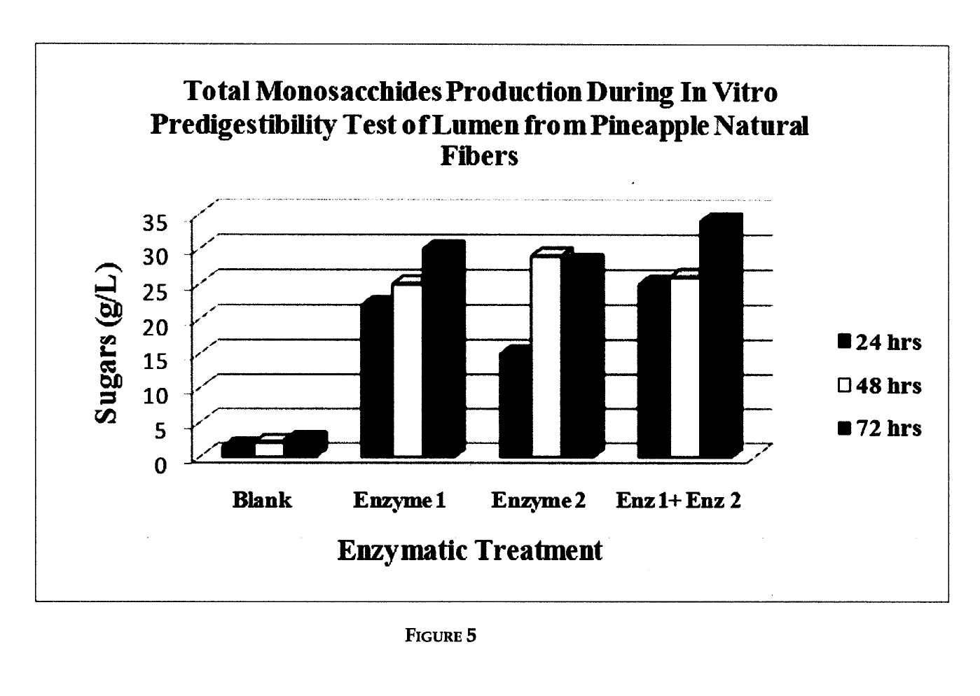

Long Fiber and Lumen, in vitro digestively results (illustrating the breakdown of fiber into monomeric sugar components). In an embodiment of the present invention, the lumen mainly and long fibers being valorized for its potential for preparing animal feeding and also with potential as organic fertilizer in both cases the possibility to supply high quality sugars and energy for growth improvement in both animal and plant systems. In a range of 15 g to 25 g, more preferable 17 g to 21 g, most preferable 17 g to 20 g dry weight of lumen or long fiber material are placed in 250 ml flasks. A buffer solution of Phosphate buffer range 5.7 to 7.0M, made 1.4M of dibasic K₂HPO₃ in 200ml of DI water where the pH is 8.75, also 0.78 monobasic KH₂PO₄ in 300ml DI water, pH 4.5, add Sodium azide 500mM to get 0.03%, more preferable 0.025%, most preferable 0.02%, keep the buffer at cool temperature between 3 to 7° C., more preferable 2 to 5° C., most preferable 4 to 8° C. The buffer solution is added to the flasks containing the lumen and long fibers respectively, also add the appropriate amount of enzyme dosages to each flask, the reactions are incubated at a controlled temperature range 45° C. to 49° C., more preferable 53° C. to 55° C., most preferable 50° C. to 60° C. with vigorous shaking range 200 rpm to 250 rpm, more preferable 275 rpm to 285 rpm, most preferable 290 rpm to 325 rpm, for the specified time 24 hours, 48 hours and 72 hours. As depicted in FIG. 5, the lumen samples were enzymatically digested under specific hydrolytic reaction conditions, to show that the combination of enzymes produces from30 g/L to 35 g/L which outperform over the untreated control samples. At 24 hours it is possible to obtain up to 30 g/L of combined sugars which is excellent for initiating the energy intake within the first 24 hours. The above results display the efficacious enzymatic digestibility of the lumen fraction and the long fibers prepared as value added products by the present invention.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method for isolating and extracting a plurality of constituents from a pineapple plant comprising
obtaining a pineapple plant;
separating said pineapple plant into at least two portions, one first portion containing pineapple plant stems and one second portion containing pineapple plant leaves, wherein each of said portions are processed to produce constituents from said pineapple plant;
isolating a plurality of constituents from each of said first portion contain pineapple plant stems and said second portion containing pineapple plant leafs.

2. The method for isolating and extracting a plurality of constituents from a pineapple plant according to claim 1 further comprising the stop of subjecting said pineapple plant to hydro-mechanical procedures to remove any dirt or foreign debris positioned on said plant.

3. The method for isolating and extracting a plurality of constituents from a pineapple plant according to claim 3 wherein said pineapple plant Smooth Cayenne, Red Spanish, Queen and Abacaxi, Hilo, Sugar Loaf, White Sugar Loaf, Kona Sugar Loaf, Natal Queen, Pernambuco, Queen, Green Spanish, Manzana, DelMonte Gold, Perolera, Maipuri, Singapore Spanish, Singapore Canning, Hawaiian Gold, Super Sweet, Ultra Sweet, MD2, or combinations thereof.

4. The method for isolating and extracting a plurality of constituents from a pineapple plant according to claim 1 wherein said stems are subjected to one or more processes to form a plurality of first stem separation fractions and said leaves are subjected to one or more processes to form a plurality of first leaf separation fractions.

5. The method for isolating and extracting a plurality of constituents from a pineapple plant according to claim 1 wherein said step of preparing said plant for isolation of one or more various plant based constituents includes subjecting said plant to hydromechanical procedures, thermomechanical procedures, extractive chemical procedures, or combinations thereof.

6. The method for isolating and extracting a plurality of constituents from a pineapple plant according to claim 1 further including the step of subjecting said leaf portions to a delamination process.

7. The method for isolating and extracting a plurality of constituents from a pineapple plant according to claim 5 wherein said delamination process separates said leaf portions into at least three components.

8. The method for isolating and extracting a plurality of constituents from a pineapple plant to claim 1 wherein said pineapple plant has been removed from the ground no longer than 24 hours.

9. The method for isolating and extracting a plurality of constituents from a pineapple plant according to claim 1 wherein said stem portion and said leaf portions are further processed to isolate additional products.

10. The method for isolating and extracting a plurality of constituents from a pineapple plant to claim 1 further including the steps of fractionation.

11. The method for isolating and extracting a plurality of constituents from a pineapple plant to claim 1 further including the steps of centrifugation.

12. The method for isolating and extracting a plurality of constituents from a pineapple plant to claim 1 wherein said pineapple plant stems are processed to provide bromelain.

13. The method for isolating and extracting a plurality of constituents from a pineapple plant to claim 1 wherein said pineapple plant stems are processed to provide one or more types of fibers.

14. The method for isolating and extracting a plurality of constituents from a pineapple plant to claim 1 wherein said pineapple plant stems are processed to provide sugars.

15. The method for isolating and extracting a plurality of constituents from a pineapple plant to claim 7 wherein said pineapple plant stems are processed to provide flour.

16. The method for isolating and extracting a plurality of constituents from a pineapple plant to claim 1 wherein said pineapple plant leaves are processed to provide fiber.

17. The method for isolating and extracting a plurality of constituents from a pineapple plant to claim 1 wherein said pineapple plant leaves are processed to provide lumen.

18. The method for isolating and extracting a plurality of constituents from a pineapple plant to claim 1 wherein said pineapple plant leaves are processed to provide paper.

19. A method for isolating and extracting a plurality of constituents from the stems of a pineapple plant comprising
obtaining stems from a pineapple plant;
subjecting said stems from a pineapple plant to a first set of procedures to form at least two first leaf separation fractions;
isolating a plurality of constituents from each said first leaf separation fractions.

20. A method for isolating and extracting a plurality of constituents from the leaves of a pineapple plant comprising
obtaining leaves from a pineapple plant;
subjecting said leaves from a pineapple plant to one or more processes to form a plurality of first leaf separation fractions;
isolating a plurality of constituents from each said first leaf separation fractions.
